(19) United States
(12) Patent Application Publication
(10) Pub. No.: US 2025/0359934 A1
(43) Pub. Date: Nov. 27, 2025

(54) MEDICAL DEVICE, ENDOSCOPE SYSTEM, CONTROL METHOD, COMPUTER-READABLE RECORDING MEDIUM, AND LEARNING DEVICE

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Kotone NAKAJIMA, Tokyo (JP)

(73) Assignee: OLYMPUS MEDICAL SYSTEMS CORP., Tokyo (JP)

(21) Appl. No.: 19/290,756

(22) Filed: Aug. 5, 2025

Related U.S. Application Data
(63) Continuation of application No. PCT/JP2023/004452, filed on Feb. 9, 2023.

(51) Int. Cl. A61B 18/26; A61B 1/00; A61B 1/04; A61B 1/06; A61B 18/00; G16H 40/63

(52) U.S. Cl. CPC A61B 18/26; A61B 1/00006; A61B 1/000096; A61B 1/043; A61B 1/0661; G16H 40/63; A61B 2018/00982; A61B 2218/002

(57) ABSTRACT

A medical device includes a processor including hardware, the processor being configured to: generate a taken image by capturing fluorescence generated from a body tissue as a result of irradiation of an excitation light on the body tissue, determine variation in state of heat denaturation based on the taken image, and send a control signal for controlling an operation of a perfusion device configured to perfuse a perfusate, to the perfusion device, based on a result of determination about the variation in the state of heat denaturation.

### CROSS REFERENCES TO RELATED APPLICATIONS

This application is a continuation of International Application No. PCT/JP2023/004452, filed on Feb. 9, 2023, the entire contents of which are incorporated herein by reference.

### BACKGROUND

### 1. Technical Field

The present disclosure relates to a medical device, an endoscope system, a control method, a computer-readable recording medium, and a learning device.

### 2. Related Art

In the related art, a technology is known that, at the time of performing heat treatment on the body tissue using an energy device, enables visualization of the state of heat denaturation of the body tissue (for example, refer to International Laid-open Pamphlet No. 2020/174666).

In the technology disclosed in International Laid-open Pamphlet No. 2020/174666, based on a taken image that captures the fluorescence generated from the body tissue as a result of irradiation of an excitation light on the body tissue, the state of heat denaturation of the body tissue is visualized. More particularly, in the technology disclosed in International Laid-open Pamphlet No. 2020/174666, from among all of the pixels of the taken image, the regions in which the fluorescence intensity is higher than the preset fluorescence intensity are displayed as the regions having high heat denaturation.

### SUMMARY

In some embodiments, a medical device includes a processor including hardware, the processor being configured to: generate a taken image by capturing fluorescence generated from a body tissue as a result of irradiation of an excitation light on the body tissue, determine variation in state of heat denaturation based on the taken image, and send a control signal for controlling an operation of a perfusion device configured to perfuse a perfusate, to the perfusion device, based on a result of determination about the variation in the state of heat denaturation.

In some embodiments, an endoscope system includes: a light source device configured to emit an excitation light; an endoscope configured to be inserted into a subject and output a taken image that is generated by capturing fluorescence generated from a body tissue inside the subject as a result of irradiation of the excitation light on the body tissue; and a medical device including a processor including hardware, the processor being configured to determine variation in state of heat denaturation based on the taken image, and send a control signal for controlling an operation of a perfusion device configured to perfuse a perfusate, to the perfusion device, based on a result of determination about the variation in the state of heat denaturation.

In some embodiments, provided is a control method implemented in a medical device. The method includes: determining variation in state of heat denaturation based on a taken image that is generated by capturing fluorescence generated from a body tissue as a result of irradiation of an excitation light on the body tissue, and sending a control signal for controlling an operation of a perfusion device configured to perfuse a perfusate, to the perfusion device, based on a result of determination about the variation in the state of heat denaturation.

In some embodiments, provided is a non-transitory computer-readable recording medium with an executable program stored thereon. The program causes a medical device to execute: determining variation in state of heat denaturation based on a taken image that is generated by capturing fluorescence generated from a body tissue as a result of irradiation of an excitation light on the body tissue, and sending a control signal for controlling an operation of a perfusion device configured to perfuse a perfusate, to the perfusion device, based on a result of determination about the variation in the state of heat denaturation.

In some embodiments, a learning device includes a processor including hardware, the processor being configured to generate a learnt model by performing machine learning using teacher data in which a fluorescence image that is generated by capturing fluorescence generated from a body tissue as a result of irradiation of an excitation light on the body tissue is treated as input data, and information corresponding to a control signal for controlling an operation of a perfusion device configured to perfuse a perfusate based on variation in state of heat denaturation as extracted from the fluorescence image is treated as output data.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to an embodiment;

FIG. 2 is a block diagram illustrating a functional configuration of the main parts of the endoscope system according to the embodiment;

FIG. 3 is a diagram illustrating the wavelength characteristics of the excitation light emitted by a second light source;

FIG. 4 is a diagram illustrating the transmission characteristics of a cut filter;

FIG. 5 is a diagram for explaining the principle of observation applied in a fluorescence observation mode;

FIG. 6 is a diagram for explaining the principle of observation applied in a normal light observation mode;

FIG. 7 is a flowchart for explaining a control method implemented by a control device; and

FIGS. 8 to 10 are diagrams for explaining the control method.

### DETAILED DESCRIPTION

An illustrative embodiment (hereinafter, called an embodiment) is described below with reference to the accompanying drawings. However, the disclosure is not limited by the embodiment described below. Moreover, in the drawings, identical constituent elements are referred to by the same reference numerals.

### Overall Configuration of Endoscope System

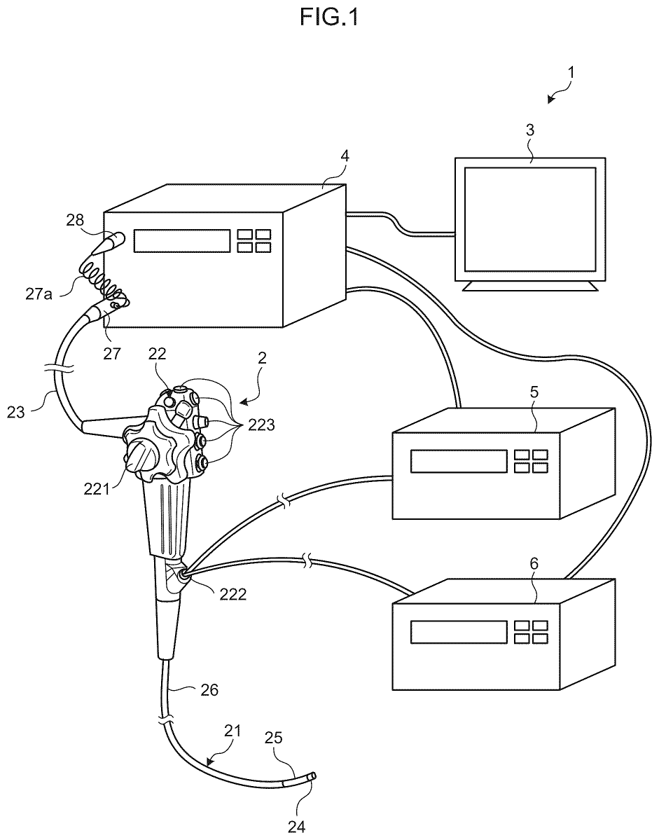

FIG. 1 is a diagram illustrating an overall configuration of an endoscope system 1 according to the embodiment.

The endoscope system 1 according to the embodiment is used in transurethral uretero-lithotripsy (TUL). More particularly, in transurethral uretero-lithotripsy, an insertion portion 21 of an endoscope 2 is inserted into the urinary tract of the subject and an in-vivo image of the subject is taken; and a display image based on the obtained image data is displayed in a display device 3. Then, while checking the display image, the operator bombards a laser from a laser irradiation device 5 toward a calculus formed inside the subject and crushes the calculus; removes the crushed calculus using a treatment tool such as a basket catheter; and places a medical device inside the urinary tract for a predetermined period of time. The medical device implies either a stent, a catheter, or an intravenous cannula.

As illustrated in FIG. 1, the endoscope system 1 includes an endoscope 2, a display device 3, a control device 4, the laser irradiation device 5, and a perfusion device 6.

The endoscope 2 generates image data (RAW data) of in-vivo images of the subject, and outputs the image data to the control device 4. As illustrated in FIG. 1, the endoscope 2 includes the insertion portion 21, an operating unit 22, and a universal cord 23.

The insertion portion 21 is at least partially flexible and is inserted inside the subject. As illustrated in FIG. 1, the insertion portion 21 includes a front end portion 24 at the front end thereof; a freely-bendable curved portion 25 that is connected to the proximal end of the front end portion 24 (i.e., the side toward the operating unit 22); and a flexible tube 26 that is a flexible and long tube connected to the proximal end of the curved portion 25.

The operating unit 22 is connected to the proximal end portion of the insertion portion 21. The operating unit 22 receives various operations performed with respect to the endoscope 2. As illustrated in FIG. 1, the operating unit 22 includes a bending knob 221, an insertion opening 222, and a plurality of operating members 223.

The bending knob 221 is configured to be rotationally movable according to a user operation performed by the user such as an operator. As a result of the rotational movement of the bending knob 221, a bending mechanism (not illustrated) that is made of a metal wire or a resin wire and that is disposed inside the insertion portion 21 is operated. With that, the curved portion 25 bends.

The insertion opening 222 is communicated with a treatment tool channel (not illustrated) that is a pipe conduit extending from the front end of the insertion portion 21, and serves as the insertion opening for inserting a treatment tool from the outside of the endoscope 2 into the treatment tool channel.

The operating members 223 are configured using buttons for receiving various operations performed by the user such as an operator; and output operation signals corresponding to the various operations to the control device 4 via the universal cord 23. Examples of the various operations include an operation for switching the observation mode of the endoscope system 1 between a normal-light observation mode and a fluorescence observation mode.

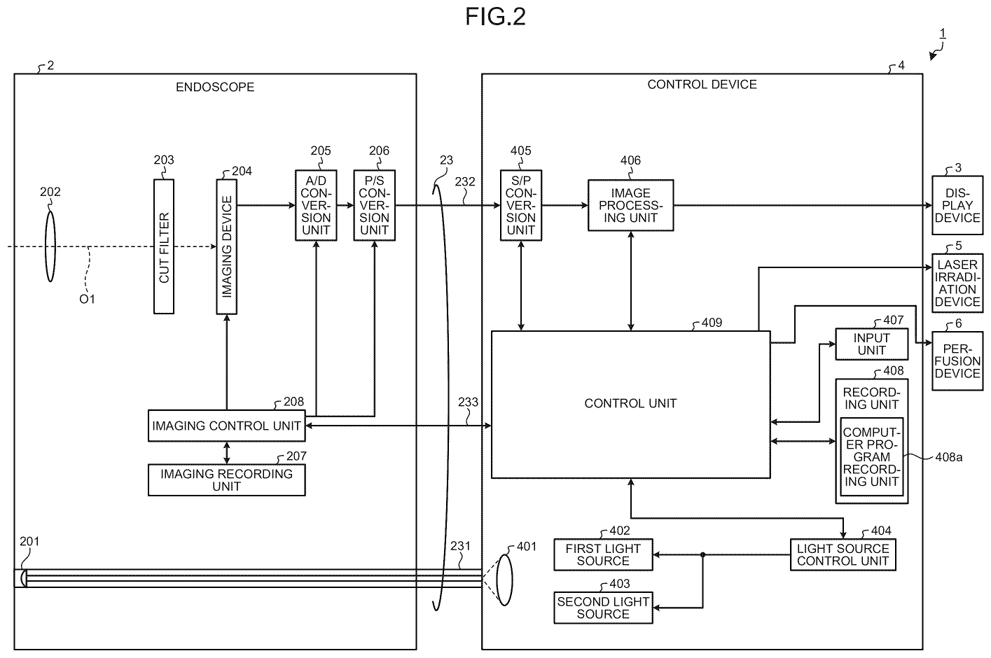

The universal cord 23 extends from the operating unit 22 in a different direction than the direction of extension of the insertion portion 21; and has a light guide 231 (see FIG. 2) made of an optical fiber arranged thereon, has a first signal line 232 (see FIG. 2) arranged thereon for transmitting the image data, and has a second signal line 233 (see FIG. 2) arranged thereon for transmitting the operation signals. Moreover, as illustrated in FIG. 1, a first connector portion 27, a second connector portion 28, and a cable 27a are disposed at the proximal end of the universal cord 23.

The first connector portion 27 is connected to the control device 4 in a detachably attachable manner.

The cable 27a is a coiled cable extending from the first connector portion 27.

The second connector portion 28 is provided at the front end of the cable 27a and is connected to the control device 4 in a detachably attachable manner.

The display device 3 is configured using a display monitor such as a liquid crystal display or an organic electroluminescence (EL) display; and, under the control performed by the control device 4, displays a display image based on the image data having been subjected to image processing in the control device 4 and displays a variety of information related to the endoscope system 1.

The control device 4 is equivalent to a medical device. The control device 4 is implemented using a processor representing a processing device equipped with hardware such as a graphics processing unit (GPU), a field programmable gate array (FPGA), or a central processing unit (CPU); and using a memory representing a temporary memory area used by the processor. According to the computer programs stored in the memory, the control device 4 comprehensively controls the operations of the constituent elements of the endoscope system 1.

The laser irradiation device 5 emits a high-output infrared laser, such as a holmium: yttrium-aluminum-garnet laser, under the control performed by the control device 4. More particularly, the laser irradiation device 5 is inserted from the insertion opening 222 into the urinary tract (for example, into a kidney, the ureter, the urinary bladder, and the urethra) via the treatment tool channel provided inside the insertion portion 21. Then, according to a user operation performed by the user such as an operator, the laser irradiation device 5 irradiates a calculus, which is formed inside the subject, with the laser. As a result, the calculus gets crashed.

The perfusion device 6 is configured using a tube or a pump and, as illustrated in FIG. 1, is communicated with the treatment tool channel of the insertion portion 21 from the insertion opening 222.

The urinary tract is filled with a perfusate such as the normal saline. Then, under the control performed by the control device 4, from the insertion opening 222 via the treatment tool channel of the insertion portion 21, the perfusion device 6 sends a perfusate into the urinary tract as well as discharges the perfusate present in the urinary tract to the outside.

### Functional Configuration of Main Parts of Endoscope System

Given below is the explanation of a functional configuration of the main parts of the endoscope system 1.

FIG. 2 is a block diagram illustrating a functional configuration of the main parts of the endoscope system 1.

The following explanation is given about the endoscope 2 and the control device 4 in that order.

### Configuration of Endoscope

Firstly, the explanation is given about a configuration of the endoscope 2.

As illustrated in FIG. 2, the endoscope 2 includes an illumination optical system 201, an imaging optical system 202, a cut filter 203, an imaging device 204, an A/D conversion unit 205, a P/S conversion unit 206, an imaging recording unit 207, and an imaging control unit 208.

The illumination optical system 201, the imaging optical system 202, the cut filter 203, the imaging device 204, the A/D conversion unit 205, the P/S conversion unit 206, the imaging recording unit 207, and the imaging control unit 208 are disposed in the front end portion 24.

The illumination optical system 201 is configured using one or more lenses; and bombards an illumination light, which is supplied from the light guide 231, toward the subject.

The imaging optical system 202 is configured using one or more lenses; and condenses the lights such as the reflected light that has reflected from the subject, the optical feedback coming from the subject, and the fluorescence emitted by the subject, and forms a subject image on the light receiving surface of the imaging device 204.

The cut filter 203 is disposed on an optical axis L1 of the imaging optical system 202 and in between the imaging optical system 202 and the imaging device 204. The cut filter 203 blocks the lights having predetermined wavelength bands and allows passage of other lights.

Regarding the transmission characteristics of the cut filter 203, the explanation is given later in the section “configuration of control device”.

The imaging device 204 is configured using a charge coupled device (CCD) or a CMOS image sensor (CMOS stands for Complementary Metal Oxide Semiconductor) in which one of the color filters constituting a Bayer layout (RGGB) is disposed in each of a plurality of pixels arranged in a two-dimensional matrix. Then, under the control performed by the imaging control unit 208, the imaging device 204 receives light of the subject image that is formed by the imaging optical system 202 and that has passed through the cut filter 203; performs photoelectric conversion to generate image data (RAW data); and outputs the image data to the A/D conversion unit 205.

The A/D conversion unit 205 is configured using an A/D conversion circuit and, under the control performed by the imaging control unit 208, performs A/D conversion with respect to the analog image data input from the imaging device 204; and outputs the post-conversion image data to the P/S conversion unit 206.

The P/S conversion unit 206 is configured using a P/S conversion circuit and, under the control of the imaging control unit 208, performs parallel/serial conversion with respect to the digital image data (a taken image) input from the A/D conversion unit 205; and outputs the post-conversion image data to the control device 4 via the first signal line 232.

Meanwhile, instead of using the P/S conversion unit 206, it is possible to use an E/O conversion unit that converts the image data into optical signals, so that the image data in the form of optical signals is sent to the control device 4. Moreover, for example, the image data can be sent to the control device 4 using wireless communication such as Wi-Fi (Wireless Fidelity) (registered trademark).

The imaging recording unit 207 is configured using a nonvolatile memory or a volatile memory, and is used to record a variety of information related to the endoscope 2 (for example, the pixel information of the imaging device 204 and the characteristics of the cut filter 203). Moreover, the imaging recording unit 207 is used to record a variety of setting data and control parameters that are sent from the control device 4 via the second signal line 233.

The imaging control unit 208 is implemented using a timing generator (TG), a processor that represents a processing device equipped with hardware such as a CPU, and a memory that represents a temporary memory area used by the processor. Based on the setting data received from the control device 4 via the second signal line 233, the imaging control unit 208 controls the constituent elements such as the imaging device 204, the A/D conversion unit 205, and the P/S conversion unit 206.

### Configuration of Control Device

Given below is the explanation of a configuration of the control device 4.

As illustrated in FIG. 2, the control device 4 includes a condenser lens 401, a first light source 402, a second light source 403, a light source control unit 404, an S/P conversion unit 405, an image processing unit 406, an input unit 407, a recording unit 408, and a control unit 409.

The condenser lens 401 condenses the lights emitted by the first light source 402 and the second light source 403, and emits the condensed light to the light guide 231.

Under the control performed by the light source control unit 404, the first light source 402 emits the white light (normal light) representing the visible light, and supplies the white light as the illumination light to the light guide 231. The first light source 402 is configured using a collimating lens, a white LED lamp (LED stands for Light Emitting Diode), and a driver.

Alternatively, as the first light source 402; a red LED lamp, a green LED lamp, and a blue LED lamp can be made to emit the lights in a simultaneous manner, so that the white light can be provided as the visible light. Still alternatively, the first light source 402 can be configured using a halogen lamp or a xenon lamp.

Under the control performed by the light source control unit 404, the second light source 403 emits an excitation light having a predetermined wavelength band, and supplies the excitation light as the illumination light to the light guide 231.

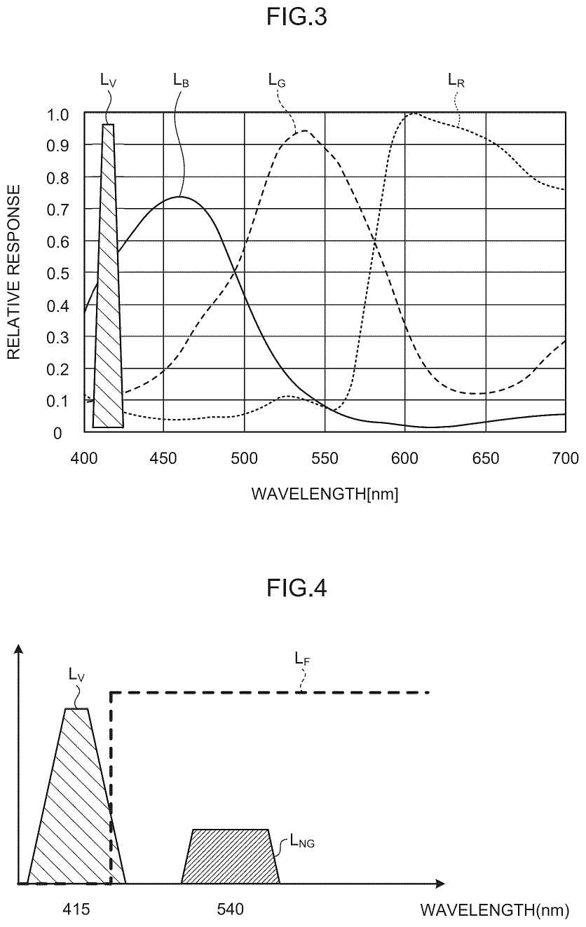

FIG. 3 is a diagram illustrating the wavelength characteristics of the excitation light emitted by the second light source 403. More particularly, in FIG. 3, the horizontal axis represents the wavelength (nm), and the vertical axis represents the wavelength characteristics. Moreover, in FIG. 3, a curved line L_{V} represents the wavelength characteristics of the excitation light emitted by the second light source 403. Furthermore, in FIG. 3, a curved line L_{B} represents the wavelength characteristics of the blue color, a curved line L_{G} represents the wavelength characteristics of the green color, and a curved line L_{R} represents the wavelength characteristics of the red color.

In the present embodiment, as illustrated in FIG. 3, the second light source 403 has the central wavelength (peak wavelength) of 415 nm, and emits an excitation light having the wavelength band between 400 nm and 430 nm. The second light source 403 is configured using a collimating lens, a semiconductor laser such as a violet laser diode (LD), and a driver.

Given below is the explanation of the transmission characteristics of the cut filter 203.

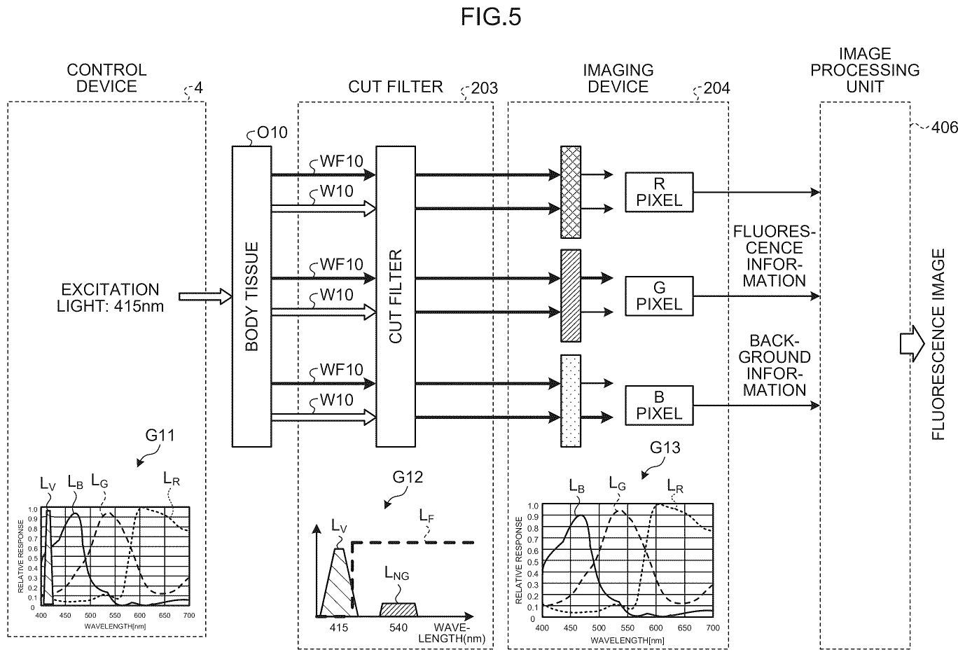

FIG. 4 is a diagram illustrating the transmission characteristics of the cut filter 203. More particularly, in FIG. 4, the horizontal axis represents the wavelength (nm) and the vertical axis represents the wavelength characteristics. Moreover, in FIG. 4, a curved line L_{F} represents the transmission characteristics of the cut filter 203, and the curved line L_{V} represents the wavelength characteristics of the excitation light. Furthermore, in FIG. 4, a curved line L_{NG} represents the wavelength characteristics of the fluorescence generated as a result of bombarding an excitation light on an advanced glycation end product that is generated as a result of performing heat treatment on the body tissue. In the present embodiment, heat treatment performed on the body tissue implies the state in which the laser bombarded from the laser irradiation device 5 on the calculus reaches the body tissue too or the state in which the laser-irradiated high-temperature calculus comes in contact with the body tissue.

In the disclosure, as illustrated in FIG. 4, the cut filter 203 blocks some of the excitation light that is reflected from the body tissue in the observation region, and allows passage of the light having other wavelength bands including the fluorescence component. More particularly, the cut filter 203 blocks some of the light that has the wavelength bands of small wavelengths between 400 nm and smaller than 430 nm and that includes the excitation light, and allows passage of the light that has the wavelength bands of longer wavelengths than 430 nm and that includes the fluorescence generated as a result of bombarding an excitation light on an advanced glycation end product generated as a result of performing heat treatment on the body tissue.

The light source control unit 404 is implemented using a processor representing a processing device equipped with hardware such as an FPGA or a CPU, and using a memory representing a temporary memory area used by the processor. Then, based on the control data input from the control unit 409, the light source control unit 404 controls the light emission timings and the light emission periods for the first light source 402 and the second light source 403.

Under the control performed by the control unit 409, the S/P conversion unit 405 performs serial/parallel conversion of the image data received from the endoscope 2 via the first signal line 232, and outputs the post-conversion image data to the image processing unit 406.

When the endoscope 2 outputs the image data using optical signals, the S/P conversion unit 405 can be substituted with an O/E conversion unit that converts the optical signals into electrical signals. Moreover, when the endoscope 2 sends the image data using wireless communication, the S/P conversion unit 405 can be substituted with a communication module capable of receiving wireless signals.

The image processing unit 406 is equivalent to a processor. The image processing unit 406 is implemented using a processor representing a processing device equipped with hardware such as a GPU or an FPGA, and using a memory representing a temporary memory area used by the processor. Under the control performed by the control unit 409, the image processing unit 406 performs predetermined image processing on the image data input as the parallel data from the S/P conversion unit 405, and outputs the resultant image data to the display device 3. Examples of the predetermined image processing include color reconstruction, white balancing, gain adjustment, y correction, and format conversion.

The input unit 407 is configured using a mouse, or a footswitch, or a keyboard, or buttons, or switches, or a touch-sensitive panel. The input unit 407 receives a user operation performed by the user such as an operator, and outputs an operation signal corresponding to the user operation to the control unit 409.

The recording unit 408 is configured using a volatile memory, or a nonvolatile memory, or a solid state drive (SSD), or a hard disk drive (HDD), or a recording medium such as a memory card. The recording unit 408 is used to record various parameters required for the operations performed in the endoscope system 1. The recording unit 408 includes a computer program recording unit 408a for recording various computer programs written to enable operations in the endoscope system 1.

The control unit 409 is equivalent to a processor. The control unit 409 is implemented using a processor representing a processing device equipped with hardware such as an FPGA or a CPU, and using a memory representing a temporary memory area used by the processor. The control unit 409 comprehensively controls the constituent elements of the endoscope system 1.

### Principle of Observation Applied in Observation Modes of Endoscope System

Given below is the explanation about the principle of observation applied in the observation modes of the endoscope system 1.

The following explanation is given about the fluorescence observation mode and the normal light observation mode in that order.

### Principle of Observation Applied in Fluorescence Observation Mode

Firstly, the explanation is given about the principle of observation applied in the fluorescence observation mode.

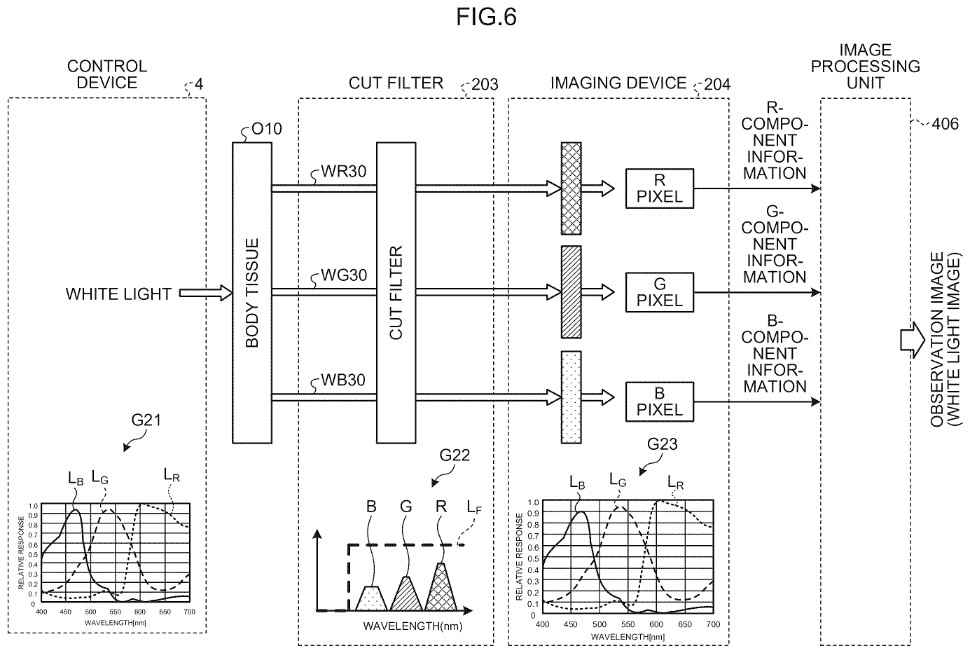

FIG. 5 is a diagram for explaining the principle of observation applied in the fluorescence observation mode.

As illustrated in a graph G11 in FIG. 5, firstly, the control device 4 causes the second light source 403 to emit a light, so that an excitation light (having the central wavelength of 415 nm) is bombarded on a body tissue O10. In that case, as illustrated in a graph G12 in FIG. 5, the reflected light (hereinafter, referred to as a reflected light W10), which at least includes the component of the excitation light reflected from the body tissue O10 and includes the return light, gets blocked by the cut filter 203 and undergoes a decrease in the intensity. On the other hand, some of the components having longer wavelengths than the wavelength band of the blocked light fall on the imaging device 204 without getting blocked.

More particularly, as illustrated in the graph G12 in FIG. 5, the cut filter 203 blocks the major portion of the reflected light W10, which falls on the G pixels in the imaging device 204 and which has the wavelength bands of small wavelengths including the wavelength band of the excitation light; and allows passage of the wavelength bands of longer wavelengths than the blocked wavelength bands. Moreover, as illustrated in the graph G12 in FIG. 5, the cut filter 203 allows passage of a fluorescence WF10 that is the autofluorescence of the advanced glycation end product generated as a result of performing heat treatment on the body tissue O10. For that reason, the reflected light W10 having a decreased intensity and the fluorescence WF10 falls on each of the R pixels, the G pixels, and the B pixels of the imaging device 204.

The G pixels of the imaging device 204 have sensitivity to the fluorescence WF10. However, as illustrated by the curved line L_{NG} having the fluorescence characteristics as illustrated in the graph G12 in FIG. 5, the fluorescence is only a minor reaction. For that reason, the output value corresponding to the fluorescence WF10 in the G pixels represents only a small value.

Then, the image processing unit 406 obtains the image data (RAW data) from the imaging device 204; performs image processing with respect to the output value of each G pixel and each B pixel included in the image data; and generates a fluorescence image. In that case, the output values of the G pixels include fluorescence information corresponding to the fluorescence WF10, which is generated from the heat treatment region (advanced glycation end product) used for performing heat treatment on the body tissue O10. The output values of the B pixels include background information obtained from the body tissue O10 of the subject in which the heat treatment region is included. When that fluorescence image is displayed in the display device 3, it becomes possible to observe the heat treatment region used for performing heat treatment on the body tissue O10.

### Principle of Observation Applied in Normal Light Observation Mode

Given below is the explanation about the principle of observation applied in the normal light observation mode.

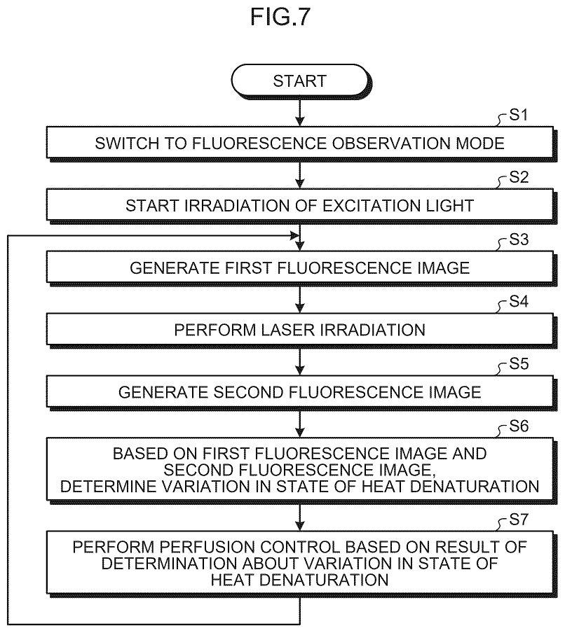

FIG. 6 is a diagram for explaining the principle of observation applied in the normal light observation mode.

As illustrated in a graph G21 in FIG. 6, firstly, the control device 4 causes the first light source 402 to emit a light, so that the white light is bombarded on the body tissue O10. In that case, of the reflected lights reflected from the body tissue O10 (hereinafter, written as reflected lights WR30, WG30, and WB30) and the return light, some light is blocked by the cut filter 203, and the remaining light falls on the imaging device 204. More particularly, as illustrated in a graph G22 in FIG. 6, the cut filter 203 blocks the reflected light having the wavelength bands of small wavelengths including the wavelength band of the excitation light. Hence, the component of the light having the wavelength band of the blue color, which falls on the B pixels of the imaging device 204, becomes smaller as compared to the state in which the cut filter 203 is not disposed.

Then, the image processing unit 406 obtains the image data (RAW data) from the imaging device 204; performs image processing with respect to the output value of each R pixel, each G pixel, and each B pixel included in the image data; and generates an observation image (white light image). In that case, since the blue color component included in the image data is smaller as compared to the state in which the cut filter 203 is not disposed, the image processing unit 406 performs white balancing for adjusting the white balance in order to ensure that the ratio of the red color component, the green color component, and the blue color component becomes constant. Thus, when the observation image (white light image) is displayed in the display device 3, it becomes possible to observe a natural observation image (white light image) regardless of whether the cut filter 203 is disposed.

### Control Method

Given below is the explanation of a control method implemented by the control device 4.

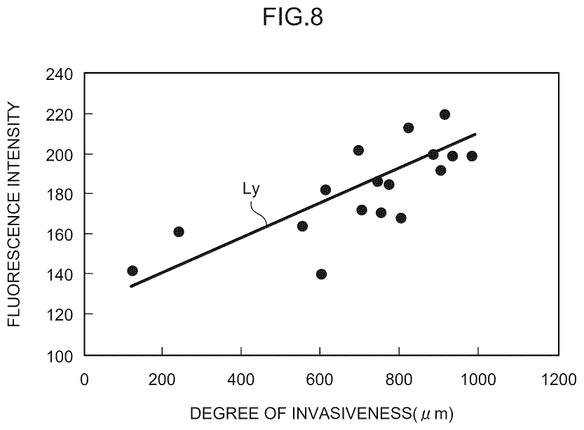

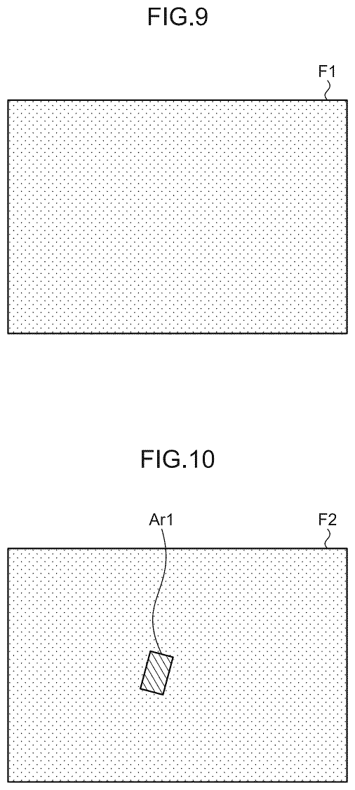

FIG. 7 is a flowchart for explaining the control method implemented by the control device 4. FIGS. 8 to 10 are diagrams for explaining the control method. FIG. 8 is a diagram illustrating the correlation (a straight line L_{Y}) between the fluorescence intensity of the autofluorescence of the advanced glycation end product in the body tissue and the degree of invasiveness (the depth and the region) into the body tissue as a result of performing heat treatment on the body tissue. In FIG. 8, the vertical axis represents the fluorescence intensity, and the horizontal axis represents the degree of invasiveness into the body tissue as a result of performing heat treatment on the body tissue. FIG. 9 is a diagram illustrating a fluorescence image (a first fluorescence image F1) that is generated at Step S3. FIG. 10 is a diagram illustrating a fluorescence image (a second fluorescence image F2) that is generated at Step S5. In FIGS. 9 and 10, the calculus that is captured in the first fluorescence image F1 and the second fluorescence image F2 is not illustrated for explanatory convenience.

The following explanation is given about the control method that is implemented by the control device 4 while transurethral uretero-lithotripsy is underway. That is, the insertion portion 21 has been inserted into the urinary tract, and accordingly the region including the calculus present inside the urinary tract represents the observation region of the endoscope system 1. Moreover, the laser irradiation device 5 has been inserted from the insertion opening 222 into the urinary tract via the treatment tool channel provided inside the insertion portion 21, and accordingly it is possible to bombard the laser on the calculus. Furthermore, the normal state is maintained in which the control unit 409 performs normal control of the perfusion device 6 so that the perfusate is filled from the insertion opening 222 into the urinary tract via the treatment tool channel provided in the insertion portion 21, and is perfused at the normal speed (i.e., while delivering the perfusate into the urinary tract, the perfusate present inside the urinary tract is discharged to the outside).

Firstly, according to an operation of “switching the observation mode of the endoscope system 1 to the fluorescence observation mode” as performed by the user such as an operator using the operating members 223, the control unit 409 switches the observation mode to the fluorescence observation mode (Step S1).

After Step S1, the control unit 409 controls the light source control unit 404 to start irradiation of the excitation light from the second light source 403 (Step S2).

After Step S2, based on the image data generated by the imaging device 204, the image processing unit 406 generates a fluorescence image (the first fluorescence image F1 (see FIG. 9)) (Step S3).

After Step S3, the control unit 409 controls the laser irradiation device 5 and causes the laser irradiation device 5 to perform laser irradiation (Step S4). As a result of the laser irradiation, the calculus present in the urinary tract gets crushed.

After Step S4, based on the image data generated by the imaging device 204, the image processing unit 406 generates a fluorescence image (the second fluorescence image F2 (see FIG. 10)) (Step S5).

Meanwhile, as illustrated in FIG. 8, the fluorescence intensity of the autofluorescence of the advanced glycation end product in the body tissue has a correlation with the degree of invasiveness (the height of heat denaturation) into the body tissue as a result of performing heat treatment on the body tissue. More particularly, as illustrated by the straight line L_{Y} in FIG. 8, greater the height of heat denaturation (i.e., greater the degree of invasiveness into the body tissue as a result of performing heat treatment on the body tissue), the greater becomes the fluorescence intensity.

In the second fluorescence image F2 illustrated in FIG. 10, a region Ar1 expressed using oblique lines represents a heat denaturation region in which heat denaturation has increased as a result of performing heat treatment on the body tissue. That is, the heat denaturation region Ar1 is equivalent to the region that gets subjected to heat treatment when the laser bombarded on the calculus at Step S4 reaches the body tissue too, or is equivalent to the region that gets subjected to heat treatment when the laser-irradiated high-temperature calculus comes in contact with the body tissue. Meanwhile, the first fluorescence image F1 illustrated in FIG. 9 is an identical image to the observation region in the second fluorescence image F2, and is taken before the laser irradiation is performed at Step S4. For that reason, in the first fluorescence image F1, a region with increased heat denaturation is absent at the position corresponding to the heat denaturation region Ar1 in the second fluorescence image F2. The first fluorescence image F1 is equivalent to a first taken image, and the second fluorescence image F2 is equivalent to a second taken image.

Depending on the degree of variation in the state of heat denaturation, there is a possibility that the body tissue gets affected.

In that regard, in the present embodiment, as a result of performing the operations at Steps S6 and S7, the effect of laser irradiation on the body tissue is handled in an appropriate manner.

More particularly, after Step S5, based on the first fluorescence image F1 and the second fluorescence image F2, the control unit 409 determines the variation in the state of heat denaturation (Step S6). In the present embodiment, based on the difference in fluorescence intensity between the corresponding pixels in the first fluorescence image F1 and the second fluorescence image F2, the control unit 409 determines the variation in the state of heat denaturation.

Examples of the fluorescence intensity used in the operation at Step S6 include at least the g value from among the pixel values (r, g, b) of each pixel of the first fluorescence image F1 and the second fluorescence image F2 that have been subjected to color reconstruction; and the luminance value corresponding to the Y signal (luminance signal).

At Step S6, although the variation in the state of heat denaturation is performed based on the first fluorescence image F1 and the second fluorescence image F2, that is not the only possible case. Alternatively, for example, the variation in the state of heat denaturation can be determined based on two sets of image data (two taken images) that are taken at different timings and that are yet to be subjected to image processing by the image processing unit 406. At that time, the output value of the G pixels in the imaging device 204 can be cited as the fluorescence intensity.

After Step S6, based on the result of determination about the variation in the state of heat denaturation as obtained at Step S6, the control unit 409 performs perfusion control (operation control of the perfusion device 6) (Step S7). Then, the system control returns to Step S3.

More particularly, operations (1) to (3) given below can be cited as the perfusion control performed at Step S7. In the following explanation, the state variation amount indicates the size of the region in which there occurs variation in the state of heat denaturation, or indicates the intensity of heat denaturation in the region in which there occurs variation in the state of heat denaturation. For example, the state variation amount determined from the first fluorescence image F1 and the second fluorescence image F2 indicates the size of the heat denaturation region Ar1 in which there occurs variation in the state of heat denaturation, or indicates the fluorescence intensity in the heat denaturation region Ar1.

(1) At Step S6, when the state variation amount indicating the variation in the state of heat denaturation is determined to be equal to or smaller than a first state variation amount, the control unit 409 continues to perform normal control of the perfusion device 6, and maintains the normal state in which the perfusate is perfusing at the normal state.

In the example of the first fluorescence image F1 and the second fluorescence image F2, the perfusion control according to the operation (1) is performed when the size of the heat denaturation region Ar1 or the fluorescence intensity of the heat denaturation region Ar1 is equal to or smaller than the first state variation amount.

(2) At Step S6, when the state variation amount indicating the variation in the state of heat denaturation is determined to be greater than the first state variation amount and is equal to or smaller than a second state variation amount that is greater than the first state variation amount, the control unit 409 sends a control signal to the perfusion device 6 and switches the perfusion control of the perfusion device 6 from the normal control to a first control. In the first control, the speed of perfusion of the perfusate is increased from the normal speed to a first speed, which is faster than the normal speed, for a first period of time. After the elapse of the first period of time, the control unit 409 sends a control signal to the perfusion device 6 and reverts the perfusion control of the perfusion device 6 to the normal control from the first control.

In the example of the first fluorescence image F1 and the second fluorescence image F2, the perfusion control according to the operation (2) is performed when the size of the heat denaturation region Ar1 or the fluorescence intensity of the heat denaturation region Ar1 is greater than the first state variation amount and is equal to or smaller than the second state variation amount.

(3) At Step S6, when the state variation amount indicating the variation in the state of heat denaturation is determined to be greater than the second state variation amount, the control unit 209 sends a control signal to the perfusion device 6 and switches the perfusion control of the perfusion device 6 from the normal control to a second control. In the second control, the speed of perfusion of the perfusate is increased from the normal speed to the first speed for a second period of time that is longer than the first period of time. After the elapse of the second period of time, the control unit 409 sends a control signal to the perfusion device 6 and reverts the perfusion control of the perfusion device 6 to the normal control from the second control.

In the example of the first fluorescence image F1 and the second fluorescence image F2, the perfusion control according to the operation (3) is performed when the size of the heat denaturation region Ar1 or the fluorescence intensity of the heat denaturation region Ar1 is greater than the second state variation amount.

Meanwhile, perfusion control according to an operation (4) can be performed in place of the perfusion control according to the operation (2); and perfusion control according to an operation (5) can be performed in place of the perfusion control according to the operation (3).

(4) At Step S6, when the state variation amount indicating the variation in the state of heat denaturation is determined to be greater than the first state variation amount and is equal to or smaller than the second state variation amount that is greater than the first state variation amount, the control unit 209 sends a control signal to the perfusion device 6 and switches the perfusion control of the perfusion device 6 from the normal control to a third control. In the third control, the speed of perfusion of the perfusate is increased from the normal speed to a second speed for a third period of time. After the elapse of the third period of time, the control unit 409 sends a control signal to the perfusion device 6 and reverts the perfusion control of the perfusion device 6 to the normal control from the third control.

(5) At Step S6, when the state variation amount indicating the variation in the state of heat denaturation is determined to be greater than the second state variation amount, the control unit 209 sends a control signal to the perfusion device 6 and switches the perfusion control of the perfusion device 6 from the normal control to a fourth control. In the fourth control, the speed of perfusion of the perfusate is increased from the normal speed to a third speed, which is faster than the second speed, for the third period of time. After the elapse of the third period of time, the control unit 409 sends a control signal to the perfusion device 6 and reverts the perfusion control of the perfusion device 6 to the normal control from the fourth control.

According to the present embodiment described above, the following effects are achieved.

In the control device 4 according to the present embodiment, the control unit 409 determines the variation in the state of heat denaturation based on the fluorescence image, and switches the perfusion control of the perfusion device 6 based on the result of determination about the variation in the state of heat denaturation.

For that reason, while transurethral uretero-lithotripsy is underway, when the laser bombarded on the calculus reaches the body tissue too or when the laser-irradiated high-temperature calculus comes in contact with the body tissue; if there occurs variation in the state of heat denaturation of the body tissue, for example, it is possible to perform the perfusion control according to the operations (2) to (5). Thus, the body tissue in which variation has occurred in the state of heat denaturation can be immediately cooled using the perfusate according to the perfusion control. Hence, the control device 4 according to the present embodiment enables performing appropriate control of the perfusion in regard to the effect of laser irradiation on the body tissue.

Moreover, in the control device 4 according to the present embodiment, based on the difference in fluorescence intensity between the corresponding pixels in the first fluorescence image F1 and the second fluorescence image F2 that are taken at different timings, the control unit 409 determines the variation in the state of heat denaturation. Then, according to the state variation amount indicating the size of the region in which there occurs variation in the state of heat denaturation or indicating the intensity of denaturation in the region in which there occurs variation in the state of heat denaturation, the control unit 409 performs the perfusion control according to the operations (1) to (5).

For that reason, when the body tissue needs to be cooled, the perfusion control according to the operations (2) to (5) can be performed, and the body tissue can be cooled in an appropriate manner.

### OTHER EMBODIMENTS

Till now, the description was given about the embodiment of the disclosure. However, the disclosure is not limited by the embodiment described above.

In the embodiment described above, the medical device is installed in an endoscope system that is used in transurethral uretero-lithotripsy. However, that is not the only possible case. Alternatively, the medical device can be installed in, for example, an endoscope system that is used in some other procedure.

In the embodiment described above, the medical device is installed in an endoscope system including a flexible endoscope. However, that is not the only possible case. Alternatively, the medical device can be installed in an endoscope system including a rigid endoscope or an endoscope system including a surgical robot.

In the embodiment described above, the control unit 409 can also function as a learning unit of a learning device. In that case, the control device 4 corresponds to the learning device.

More particularly, the control unit 409 generates a learnt model by performing machine learning using teacher data in which a fluorescence image that is generated by capturing the fluorescence generated from the body tissue as a result of bombarding the excitation light on the body tissue is treated as the input data, and the information corresponding to a control signal, which controls the operation of the perfusion device 6 based on the variation in the state of heat denaturation as extracted from the fluorescence image, is treated as the output data.

The learnt model is made up of a neural network in which each layer includes one or more nodes. There is no particular restriction of the type of machine learning. Thus, for example, it serves the purpose when teacher data, in which fluorescence images of a plurality of subjects are associated to the information corresponding to the control signals that control the operation of the perfusion device 6 based on the variation in the state of heat denaturation as extracted from the fluorescence images, is provided along with learning data; and the teacher data and the learning data is input to a calculation model that is based on a multilayered neural network, and accordingly learning is performed. Moreover, as the method for machine learning, for example, a method is implemented that is based on a convolutional neural network (CNN) or a deep neural network (DNN) of a multilayered neural network such as 3D-CNN. Alternatively, as the method for machine learning, a method can be implemented that is based on a recurrent neural network (RNN) or long short-term memory (LSTM) units obtained by expanding a recurrent neural network. Meanwhile, the functions explained above can be implemented by the learning unit of some other learning device that is different from the control device 4.

The medical device, the endoscope system, the control method, the computer program product, and the learning device according to the disclosure enable performing appropriate control of the perfusion in regard to the effect of laser irradiation on the body tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to:
generate a taken image by capturing fluorescence generated from a body tissue as a result of irradiation of an excitation light on the body tissue,
determine variation in state of heat denaturation based on the taken image, and
send a control signal for controlling an operation of a perfusion device configured to perfuse a perfusate, to the perfusion device, based on a result of determination about the variation in the state of heat denaturation.

2. The medical device according to claim 1, wherein the processor is further configured to send the control signal for increasing speed of perfusion of the perfusate, to the perfusion device, based on the result of determination about the variation in the state of heat denaturation.

3. The medical device according to claim 1, wherein the processor is further configured to send the control signal for increasing speed of perfusion of the perfusate for a first period of time, to the perfusion device, based on the result of determination about the variation in the state of heat denaturation.

4. The medical device according to claim 1, wherein the processor is further configured to determine the variation in the state of heat denaturation from two of taken images that are taken at different timings.

5. The medical device according to claim 4, wherein the two of the taken images include a first taken image that is taken before irradiation of a laser, and a second taken image that is taken after the irradiation of the laser.

6. The medical device according to claim 4, wherein the processor is further configured to determine the variation in the state of heat denaturation based on a difference in fluorescence intensity between corresponding pixels in the two of the taken images.

7. The medical device according to claim 1, wherein when it is determined that a state variation amount indicating the variation in the state of heat denaturation is greater than a first state variation amount and is equal to or smaller than a second state variation amount that is greater than the first state variation amount, the processor is further configured to send the control signal for increasing speed of perfusion of the perfusate to a first speed for a first period of time, to the perfusion device.

8. The medical device according to claim 7, wherein when it is determined that the state variation amount is greater than the second state variation amount, the processor is further configured to send the control signal for increasing speed of perfusion of the perfusate for a second period of time that is longer than the first period of time, to the perfusion device.

9. The medical device according to claim 1, wherein when it is determined that a state variation amount indicating the variation in the state of heat denaturation is greater than a first state variation amount and is equal to or smaller than a second state variation amount that is greater than the first state variation amount, the processor is further configured to send the control signal for increasing speed of perfusion of the perfusate to a second speed for a third period of time, to the perfusion device.

10. The medical device according to claim 9, wherein when it is determined that the state variation amount is greater than the second state variation amount, the processor is further configured to send the control signal for increasing speed of perfusion of the perfusate to a third speed for the third period of time, to the perfusion device, the third speed being faster than the second speed.

11. The medical device according to claim 7, wherein the state variation amount indicates size of a region in which the variation in the state of heat denaturation has occurred or indicates an intensity of the heat denaturation in a region in which the variation in the state of heat denaturation has occurred.

12. The medical device according to claim 1, wherein the fluorescence is generated from an advanced glycation end product that is generated as a result of performing a heat treatment on the body tissue.

13. An endoscope system comprising:
a light source device configured to emit an excitation light;
an endoscope configured to be inserted into a subject and output a taken image that is generated by capturing fluorescence generated from a body tissue inside the subject as a result of irradiation of the excitation light on the body tissue; and
a medical device comprising a processor comprising hardware, the processor being configured to
determine variation in state of heat denaturation based on the taken image, and
send a control signal for controlling an operation of a perfusion device configured to perfuse a perfusate, to the perfusion device, based on a result of determination about the variation in the state of heat denaturation.

14. A control method implemented in a medical device, the method comprising:
determining variation in state of heat denaturation based on a taken image that is generated by capturing fluorescence generated from a body tissue as a result of irradiation of an excitation light on the body tissue, and
sending a control signal for controlling an operation of a perfusion device configured to perfuse a perfusate, to the perfusion device, based on a result of determination about the variation in the state of heat denaturation.

15. A non-transitory computer-readable recording medium with an executable program stored thereon, the program causing a medical device to execute:
determining variation in state of heat denaturation based on a taken image that is generated by capturing fluorescence generated from a body tissue as a result of irradiation of an excitation light on the body tissue, and
sending a control signal for controlling an operation of a perfusion device configured to perfuse a perfusate, to the perfusion device, based on a result of determination about the variation in the state of heat denaturation.

16. A learning device comprising a processor comprising hardware, the processor being configured to generate a learnt model by performing machine learning using teacher data in which a fluorescence image that is generated by capturing fluorescence generated from a body tissue as a result of irradiation of an excitation light on the body tissue is treated as input data, and information corresponding to a control signal for controlling an operation of a perfusion device configured to perfuse a perfusate based on variation in state of heat denaturation as extracted from the fluorescence image is treated as output data.
